(19) United States
(12) Patent Application Publication
(10) Pub. No.: US 2008/0076772 A1
(43) Pub. Date: Mar. 27, 2008

(54) Anti-Inflammatory Agents

(75) Inventors: Paola Allavena, Milan (IT); Maurizio D'Incalci, Milan (IT); Glynn Thomas Faircloth, Cambridge, MA (US)

Correspondence Address: KING & SPALDING, 1185 AVENUE OF THE AMERICAS, NEW YORK, NY, 10036-4003, US

(73) Assignee: Pharma Mar S.A. Sociedad Unipersonal, Madrid (ES)

(21) Appl. No.: 11/576,115

(22) PCT Filed: Sep. 28, 2005
(86) PCT No.: PCT/GB05/50164

§ 371 Date: May 14, 2007

(30) Foreign Application Priority Data
Sep. 29, 2004 (US) 60614093

(51) Int. Cl. A61K 31/4995; A61P 29/00

(52) U.S. Cl. 514/250

(57) ABSTRACT

We have found anti-inflammatory activity in the ecteinascidin compounds. Such compounds have been widely described, and may have the following general formula (I):

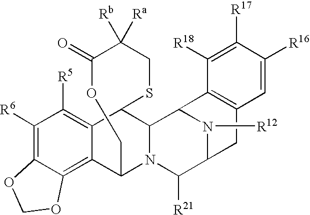

wherein:
R⁵ is OH, alkoxy or alkanoyloxy;
R⁶ is hydrogen, alkyl, alkenyl, alkynyl or aryl;
R¹² is hydrogen, alkyl, alkenyl, alkynyl or aryl;
R¹⁶ is hydrogen, alkyl, alkenyl, alkynyl or aryl;
R¹⁷ is OH, alkoxy or alkanoyloxy;
R¹⁸ is OH, alkoxy or alkanoyloxy;
R²¹ is H, OH, CN or another nucleophilic group; and
R^{a} is hydrogen and R^{b} is optionally substituted amino, or
R^{a} with R^{b} form a carbonyl function ═O, or
R^{a}, R^{b} and the carbon to which they are attached form a tetrahydroisoquinoline group.

The present invention relates to anti-inflammatory agents. More particularly, the present invention relates to the discovery of anti-inflammatory activity in a known class of compounds.

### BACKGROUND OF THE INVENTION

Monocyte/macrophages are recognized important components of innate and adaptive immunity. Circulating monocytes are versatile precursors with the ability to differentiate into the various forms of tissue macrophages. Macrophages stand guard against foreign invaders and are able to instantly defend the body against pathogens, as well as send signals for recruitment of other immunocompetent cells and present antigen to T lymphocytes. On the other hand, macrophages have also been implicated in the onset or progression of several diseases, mainly via their production of pro-inflammatory and proangiogenic mediators. Such conditions include, for instance, the pronounced inflammation present in several chronic diseases (e.g.: rheumatoid arthrites, atherosclerosis, lupus erythematosus) and tumours.

At the tumour site, Tumour-Associated Macrophages (TAM) represent a major component of infiltrating stromal cells. TAM have a complex ambiguous role within tumours, as suggested in the macrophage balance hypothesis. In fact, although macrophages stimulated with LPS and IFN gamma (also called M1 macrophages or classically activated macrophages) have the potential to kill tumour cells, several lines of evidence support the idea that macrophages within the tumour microenvironment are skewed towards alternatively activated macrophages, or M2 macrophages. Most frequently TAM are non-cytotoxic and produce several growth and angiogenic factors. TAM produce also immunosuppressive molecules (e.g. IL-10, TGFb) and a variety of inflammatory mediators, including chemokines. Chemokines activate matrix metalloproteases which digest matrix proteins and promote tumour dissemination. Thus, the accumulation of TAM at the tumour site and the continuous expression of inflammatory molecules may actually favour tumour progression.

### SUMMARY OF THE INVENTION

Ecteinascidin compounds include natural and synthetic compounds. They possess a fused five ring system, and a 1, 4 bridge. We have found anti-inflammatory activity in the ecteinascidin compounds. Such compounds have been widely described, and may have the following general formula (I):

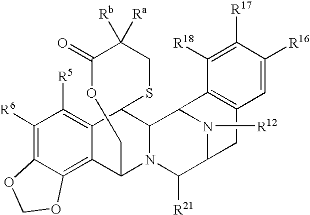

wherein:
R⁵ is OH, alkoxy or alkanoyloxy;
R⁶ is hydrogen, alky, alkenyl, alkynyl or aryl;
R¹² is hydrogen, alky, alkenyl, alkynyl or aryl;
R¹⁶ is hydrogen, alky, alkenyl, alkynyl or aryl;
R¹⁷ is OH, alkoxy or alkanoyloxy;
R¹⁸ is OH, alkoxy or alkanoyloxy;
R²¹ is H, OH, CN or another nucleophilic group; and
R^{a} is hydrogen and R^{b} is optionally substituted amino, or
R^{a} with R^{b} form a carbonyl function ═O, or
R^{a}, R^{b} and the carbon to which they are attached form a tetrahydroisoquinoline group.

Thus, the present invention provides a method of treating inflammation which comprises administration of an effective amount of an ecteinascidin having a general formula (I).

The invention also provides medicaments comprising an ecteinascidin having a general formula (I), together with a pharmaceutically acceptable carrier or diluent.

The invention further provides the use of an ecteinascidin having a general formula (I) in the preparation of a medicament for use in the treatment of inflammation.

### DETAIL DESCRIPTION OF THE INVENTION

We have found that ecteinascidin compounds posses anti-inflammatory activity. Thus, the present invention relates to a new medical indication for compounds of general formula (I) as defined above.

In these compounds the substituents can be selected in accordance with the following guidance:

Alkyl and alkoxy groups preferably have from 1 to 12 carbon atoms. One more preferred class of alkyl and alkoxy groups has from 1 to about 6 carbon atoms, and most preferably 1, 2, 3 or 4 carbon atoms. Methyl, ethyl and propyl including isopropyl are particularly preferred alkyl groups in the compounds of the present invention. Methoxy, ethoxy and propoxy including isopropoxy are particularly preferred alkyl groups in the compounds of the present invention. Another more preferred class of alkyl and alkoxy groups has from 4 to about 12 carbon atoms, yet more preferably from 5 to about 8 carbon atoms, and most preferably 5, 6, 7 or 8 carbon atoms. As used herein, the term alkyl, unless otherwise modified, refers to both cyclic and noncyclic groups, although cyclic groups will comprise at least three carbon ring members.

Preferred alkenyl and alkynyl groups in the compounds of the present invention have one or more unsaturated linkages and from 2 to about 12 carbon atoms. One more preferred class of alkenyl or alkynyl groups has from 2 to about 6 carbon atoms, and most preferably 2, 3 or 4 carbon atoms. Another more preferred class of alkenyl or alkynyl groups has from 4 to about 12 carbon atoms, yet more preferably from 5 to about 8 carbon atoms, and most preferably 5, 6, 7 or 8 carbon atoms. The terms alkenyl and alkynyl as used herein refer to both cyclic and noncyclic groups.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Specially preferred aryl groups include substituted or unsubstituted phenyl, naphthyl, biphenyl, phenanthryl and anthracyl.

Suitable alkanoyloxy and alkanoyl groups have from 2 to about 20 carbon atoms, more preferably from 2 to about 8 carbon atoms, still more preferably from 2 to about 6 carbon atoms, even more preferably 2 carbon atoms. Another preferred class of alkanoyloxy groups has from 12 to about 20 carbon, yet more preferably from 14 to about 18 carbon atoms, and most preferably 15, 16, 17 or 18 carbon atoms.

The groups above mentioned may be substituted at one or more available positions by one or more suitable groups such as OR′, ═O, SR′, SOR′, SO₂R′, NO₂, NHR′, N(R′)₂, ═N—R′, NHCOR′, N(COR′)₂, NHSO₂R′, CN, halogen, C(═O)R′, CO₂R′, OC(═O)R′ wherein each of the R′ groups is independently selected from the group consisting of H, OH, NO₂, NH₂, SH, CN, halogen, ═O, C(═O)H, C(═O)CH₃, CO₂H, substituted or unsubstituted C₁-C₁₂ alkyl, substituted or unsubstituted C₂-C₁₂ alkenyl, substituted or unsubstituted C₂-C₁₂ alkynyl and substituted or unsubstituted aryl. Suitable halogen substituents in the compounds of the present invention include F, Cl, Br and I.

Preferred compounds of the invention are those of general formula (I) wherein one or more of the following definitions will apply:
R⁵ is an alkanoyloxy;
R⁶ is methyl;
R¹² is methyl;
R¹⁶ is methyl;
R¹⁷ is methoxy;
R¹⁸ is OH;
R²¹ is H, OH or CN; and
R^{a} is hydrogen and R^{b} is an amido group, or
R^{a} with R^{b} form ═O, or
R^{a}, R^{b} and the carbon to which they are attached form a group of formula (II):

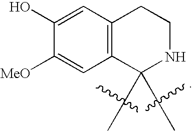

Examples of compounds for the present invention include natural ecteinascidins, such as ecteinascidin 743 and other 1,4 bridged fused ecteinascidin compounds disclosed for example in U.S. Pat. No. 5,089,273, U.S. Pat. No. 5,478,932, U.S. Pat. No. 5,654,426, U.S. Pat. No. 5,721,362, U.S. Pat. No. 6,124,293, U.S. Pat. No. 5,149,804, U.S. Pat. No. 09/ 546,877, U.S. Pat. No. 5,985,876 and WO 01/77115.

Ecteinascidin 743, also known as ET743 or ecteinascidin 743 is particularly preferred. ET743 is a natural product derived from the marine tunicate Ecteinascidinia turbinata, with potent anti-tumor activity. It is a novel effective drug that is currently in clinical trials and has shown anti-cancer activity in some human solid tumors, including soft tissue sarcomas, breast and ovarian cancer.

Compounds of the following formula (III) are particularly preferred:

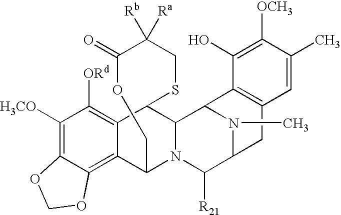

where
R^{a} is hydrogen and R^{b} is amido of formula —NHR^{f}— where R^{f} is alkanoyl, or
R^{a} with R^{b} form ═O, or
R^{a}, R^{b} and the carbon to which they are attached form a group of formula (II):

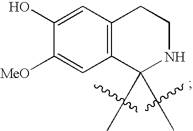

R^{d} is alkanoyl; and
R²¹ is H, OH or CN.

The alkanoyl groups can be acetyl or higher, for example up to C₂₀.

Thus, preferred compounds of this invention include:

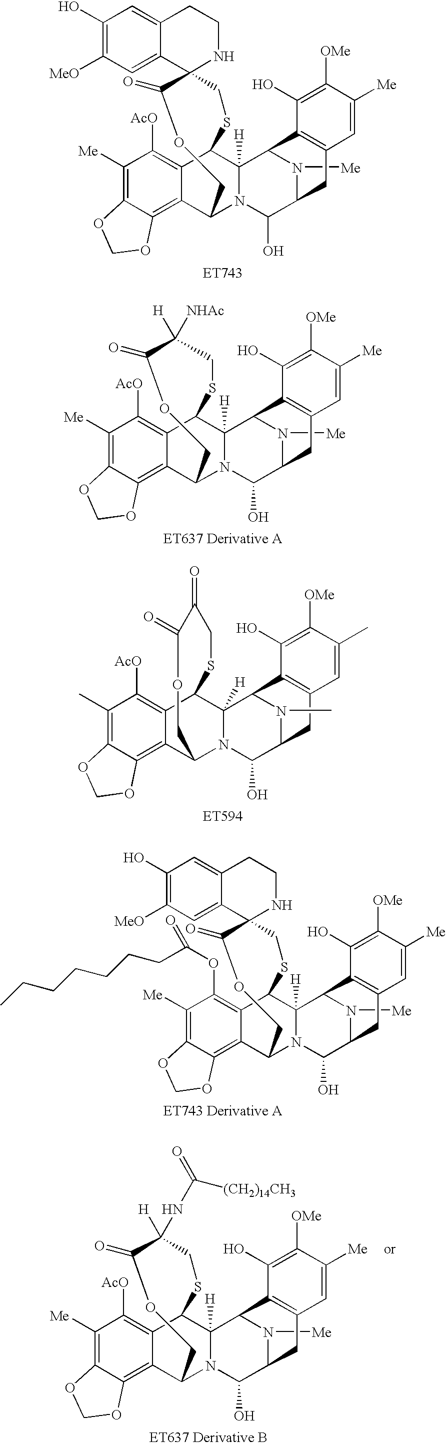

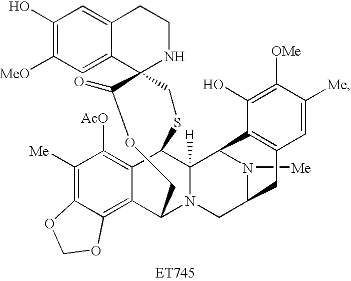

and related compounds with different acyl groups.

The medicaments provided by this invention are pharmaceutical compositions comprising the ecteinascidin compound and a pharmaceutically acceptable carrier. Medicaments can be of conventional form, and suitable dosing procedures can be devised.

As it has been indicated, the compounds of the invention are useful as anti-inflammatory agents. Thus, these compounds can be used in the treatment of diseases that deal with inflammation, particularly in the treatment of chronic inflammatory and autoimmune diseases (e.g. rheumatoid arthritis, Sjogren disease, Crohn disease) and for atherosclerosis.

### DRAWINGS

FIG. 1. Panel A: Cell viability of blood monocytes, lymphocytes and thymocytes cultured with ecteinascidin 743.

FIG. 1. Panel B: Apoptosis of monocytes treated with ecteinascidin 743.

FIG. 2. Pre-treatment with M-CSF partially protects monocytes from the pro-apoptotic effect of ecteinascidin 743.

FIG. 3. Panel A: Kinetics of the cytotoxic effect of ecteinascidin 743 on monocytes.

FIG. 3. Panel B: Inhibition of macrophage differentiation.

FIG. 4. Panel A: Susceptibility to ET743 of monocytes and macrophages from the same donor.

FIG. 4. Panel B: Susceptibility to ET743 of macrophages classically activated by LPS and IFNgamma or by IL-4.

FIG. 4. Panel C: Susceptibility to ET743 of Tumour-Associated Macrophages (TAM).

FIG. 5. In vivo infusion of ecteinascidin 743 in tumour patients induces transient monocytopenia.

FIG. 6. Ecteinascidin 743 inhibits CCL2 (Panel A) and IL-6 (Panel B) production by monocytes and macrophages.

FIG. 7. Ecteinascidin 743 inhibits CCL2 (Panel A) and IL-6 (Panel B) production in TAM and in freshly isolated tumour cells.

FIG. 8. Panel A: Ecteinascidin 743 does not affect TNF production by monocytes, macrophages and TAM.

FIG. 8. Panel B: Real time-PCR of CCL2 and TNF transcripts in LPS-stimulated monocytes exposed to ecteinascidin 743.

FIG. 9. Panel A: Cytotoxicity of ecteinascidin 743, Doxorubicin, Taxol and Cis-DDP on monocytes. The asterisc indicates the IC50 for each drug on in vitro cultured tumour cell lines.

FIG. 9. Panel B: CCL2 and TNF production by LPS-stimulated monocytes treated with the indicated doses of anti-tumour agents.

FIG. 10. CCL2 secretion by LPS-monocytes pre-treated with ecteinascidin 743 and other ecteinascidin compounds.

### EXAMPLES OF THE INVENTION

In this study we demonstrate that, at concentrations within the pharmacological range, ecteinascidin 743 showed selective toxicity for the myeloid lineage and induced apoptosis of monocyte/macrophages. At non cytotoxic concentrations ecteinascidin 743 significantly inhibited in vitro macrophage differentiation and reduced the production of selected inflammatory cytokines. These findings may be relevant for therapeutic approaches aimed at targeting monocyte/macrophages in several human diseases.

In addition to ET743, ET637 Derivative A, ET637 Derivative B, ET594, ET743 Derivative A and ET745 were also tested. They have also been shown to reduce the production of selected inflammatory cytokines.

### Materials and Methods

### Cell Preparation:

Purified populations of human blood monocytes were prepared as previously described by differential density centrifugation on Ficoll and Percoll gradients (see Allavena, P., Piemonti, L., Longoni, D., Bernasconi, S., Stoppacciaro, A., Ruco, L., and Mantovani, A. IL-10 prevents the differentiation of monocytes to dendritic cells but promotes their maturation to macrophages. Eur J Immunol, 28: 359-369, 1998). Monocytes were usually >85% CD14+ cells. Purified T lymphocytes (>95% CD3+) were obtained on Percoll gradients as previously described (see Chieppa, M., Bianchi, G., Doni, A., Del Prete, A., Sironi, M., Laskarin, G., Monti, P., Piemonti, L., Biondi, A., Mantovani, A., Introna, M., and Allavena, P. Cross-linking of the mannose receptor on monocyte-derived dendritic cells activates an anti-inflammatory immunosuppressive program. J Immunol, 171: 4552-4560, 2003). Human thymocytes were isolated from resected thymus from pediatric patients undergoing surgery. Thymocytes were obtained by teasing and isolated on Percoll gradient.

Cells were cultured at 106 cells/ml in complete medium RPMI (Biochrom, Berlin, FRG)+10% FCS (Hyclone, Logan, Utah). In vitro differentiated macrophages were obtained by culture of monocytes Monocyte-Colony Stimulating Factor (M-CSF) Peprotech (20 ng/ml), for 5 days. In some experiments, macrophages were treated with LPS (100 ng/ml) Sigma Aldrich, IFN gamma (500 IU/ml) or IL-4 (20 ng/ml) (Schering Plough) for 24 h.

Tumour-associated macrophages (TAM) and tumour cells were isolated from the ascitic fluid of patients with diagnosed ovarian adenocarcinoma, admitted to the Clinic of Obstetrics and Gynecology of the University of Milan-Bicocca, S Gerardo Hospital. Cells contained in the ascitic fluid were centrifuged and isolated by differential density gradients of Ficoll and Percoll, and plastic adherence as previously described (see Allavena, P., Peccatori, F., Maggioni, D., Erroi, A., Sironi, M., Colombo, N., Lissoni, A., Galazka, A., Meiers, W., Mangioni, C., et al. Intraperitoneal recombinant gamma-interferon in patients with recurrent ascitic ovarian carcinoma: modulation of cytotoxicity and cytokine production in tumour-associated effectors and of major histocompatibility antigen expression on tumour cells. Cancer Res, 50: 7318-7323, 1990). Purity of TAM and tumour cell preparations was usually>65±10% as defined by morphology and phenotype analysis. Cells were treated with ecteinascidin 743 at the indicated concentrations and cultured for 1-5 days, as specified in figure legends. At the end of the incubation period cells were collected, washed and used for DNA analysis or functional assays.

### Determination of Cell Viability.

Cell viability was analyzed by DNA content in Flow Cytometry

Cells exposed to treatments were fixed with ethanol 70%, washed in PBS and stained with propidium iodide (PI) solution containing 10 ug/ml PI in PBS and 25 μl RNAse 10,000 units, overnight in the dark. PI incorporation was evaluated on at least 20,000 cells/sample using a FACS Calibur instrument (Becton Dickinson, Sunnyvale, Calif., USA), with a bandpass filter at 620 nm. Apoptosis was detected by staining with AnnexinV and PI. FACS analysis was performed using a bandpass filter 530 and 620 nm for green (AnnexinV) and red (PI) fluorescence respectively, in combination with a 570 nM dichroic mirror.

### Phenotype Analysis.

Expression of cell membrane markers was performed by immunofluoresce and analyzed by Flow Cytometry. Cells were incubated with anti-CD14, anti-CD16, anti-CD68, anti-CD206 (mannose receptor) and then with FITC-goat anti-mouse Ig as described. At least 10,000 cells were analyzed.

### Cytokine Production.

Supernatants of untreated cells or cells treated with ecteinascidin 743 or other anti-neoplastic agents were collected after 24 h culture and frozen. Monocytes, macrophages and TAM were stimulated with 100 ng/ml LPS to induce maximal cytokine production. Determination of cytokines CCL2, TNF and IL-6 was measured by specific ELISA following the manufacturer's instructions.

### Tumour Patients.

Patients with sarcoma or ovarian cancer undergoing Phase II trial with ecteinascidin 743, were admitted to the European Oncology institute, Milano, Italy. Patients received ecteinascidin 743 (1300 mg/m2) in a 3-h infusion. Blood samples (40 ml) were collected immediately before the treatment and at the end of the infusion (+3 h). Blood samples were immediately processed and Percoll purified monocytes (usually 106 cells) were cultured with M-CSF (20 ng/ml) for 5 days. Differentiated cells were harvested, counted and analyzed for phenotype expression, as described above. Results are presented as absolute numbers of marker-positive cells/10,000 cells. Significant inhibition of macrophage differentiation was considered a 50% reduction of marker+cells, relative to cells collected before therapy, from the same patient.

### EXAMPLE 1

### Ecteinascidin 743 Shows Selective Cytotoxic Effect on Mononuclear Phagocytes

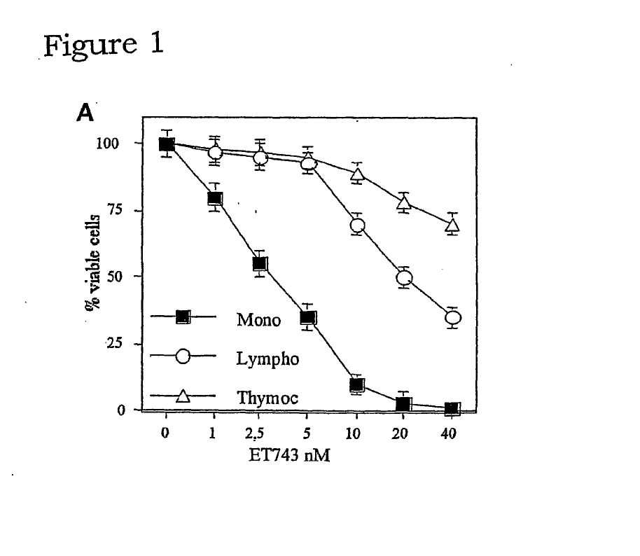

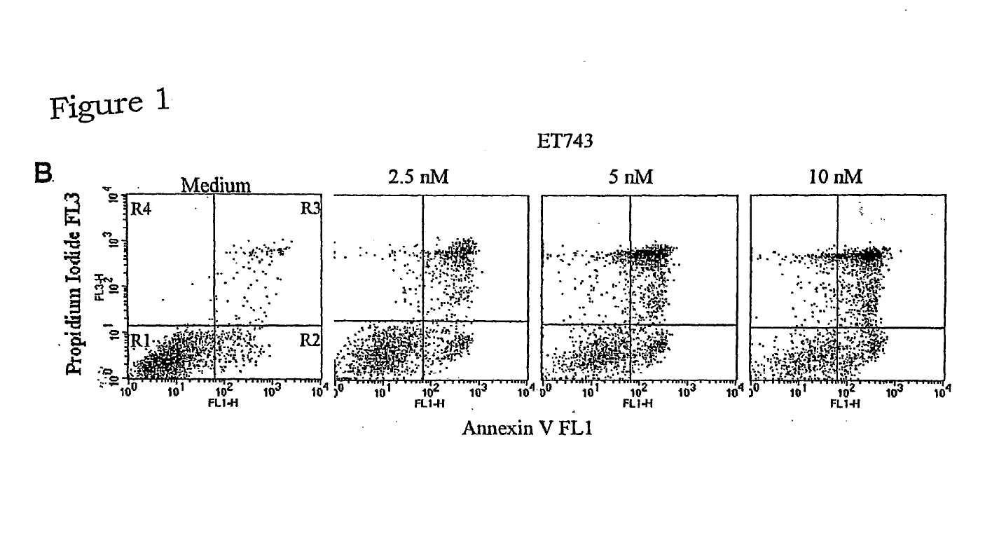

We first studied the effect of ecteinascidin 743 treatment on the viability of human leukocyte subsets in vitro. Purified preparations of blood monocytes, lymphocytes and thymocytes were cultured with different concentrations of ecteinascidin 743 for 48 h. Cell viability was assessed by DNA analysis and propidium iodide (PI) staining in Flow cytometry. Purified preparations of blood monocytes were highly susceptible to the cytotoxic effect of the drug. There was a dose-dependent mortality with a lethal dose 50% (IC50) of 2.5-5 nM after 48 h of culture (FIG. 1A). Purified T lymphocytes were much less susceptible and at 5 nM were all alive. IC50 for lymphocytes was 20 nM. Even more resistant were freshly isolated thymocytes (IC50>40 nM, FIG. 1A).

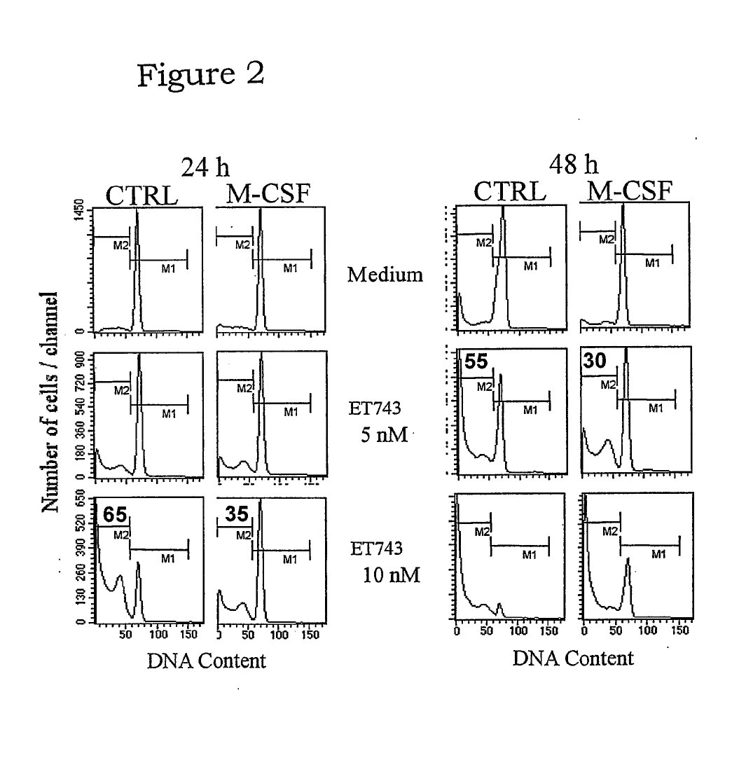

Virtually all dying monocytes exposed to ecteinascidin 743 stained positive for Annexin V, indicating that the drug induces apoptosis (FIG. 1B). Monocyte mortality was confirmed also by DNA analysis in Flow Cytometry (FIG. 2). In the presence of M-CSF, a growth and differentiation factor for monocytes, a partial protection from the toxic effect of ecteinascidin 743 was observed. M-CSF shifted monocyte death from 55% to 30% at 5 nM ecteinascidin 743, after 48 h incubation, and from 65% to 35% at 10 nM, after 24 h treatment (FIG. 2). M-CSF was effective only if added simultaneously or before ecteinascidin 743, but was no longer effective when given 4 h after the drug.

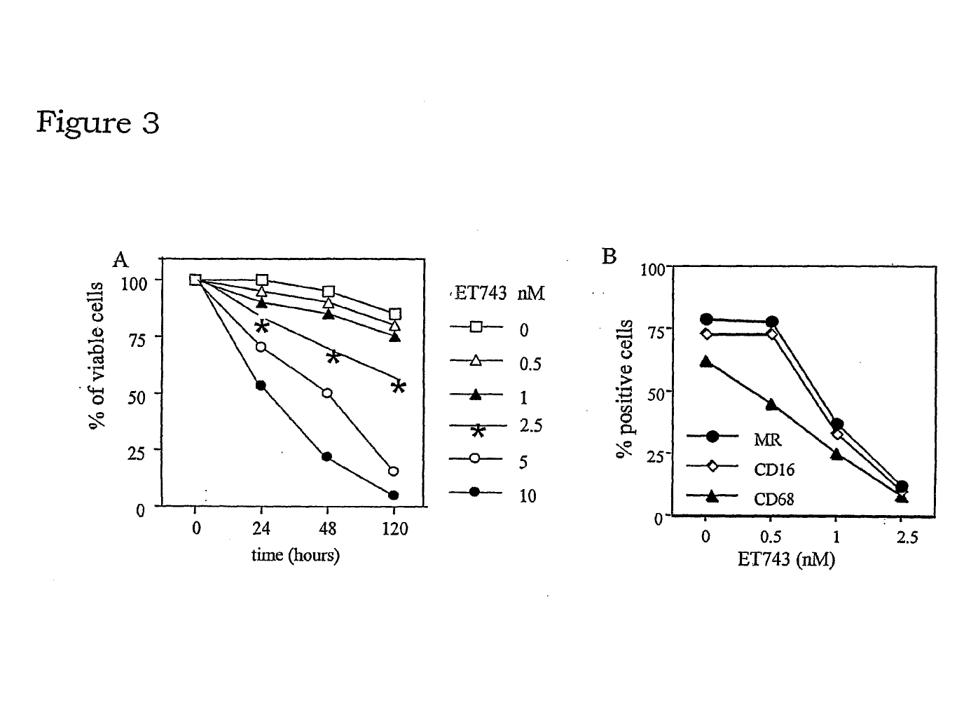

A kinetics analysis of the cytotoxic effect of ecteinascidin 743 was performed in the presence of M-CSF. Cells were treated with M-CSF (20 ng/ml) and different concentrations of ecteinascidin 743. Samples were collected at the indicated times and tested for DNA analysis. At higher concentrations, significant toxicity was observed already after 24 h incubation and increased over time (FIG. 3A). Lower concentrations (2.5 nM) induced 40-50% mortality after 5 days.

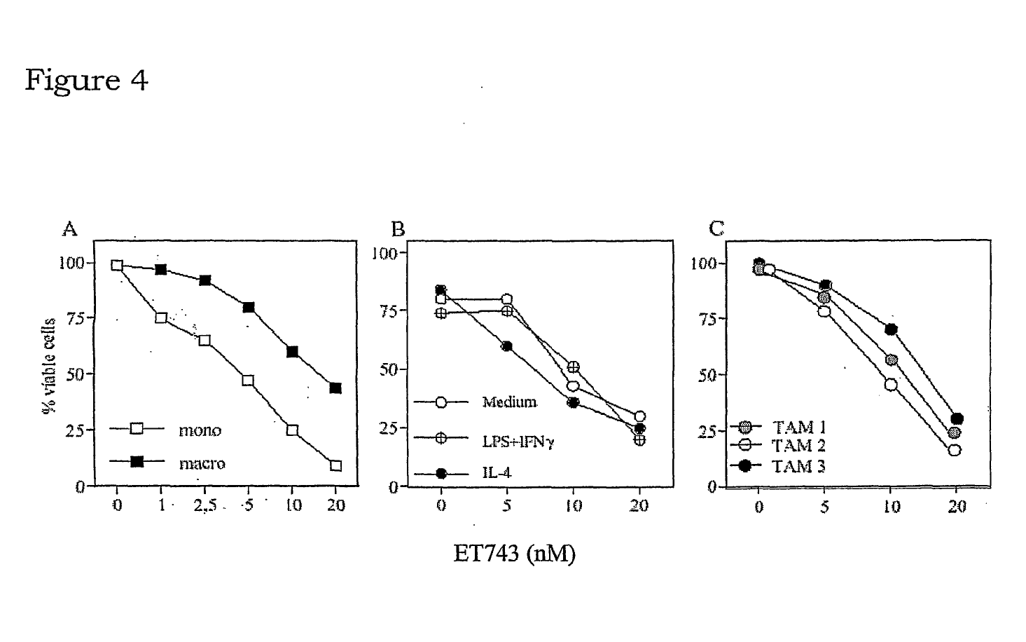

We next studied the effect of ecteinascidin 743 on already differentiated macrophages obtained from monocytes cultured in vitro for 5 days with M-CSF. The addition of ecteinascidin 743 in the last 48 h resulted in significant mortality, but to a lower extent compared to freshly isolated monocytes. FIG. 4A shows a representative experiment comparing the susceptibility of monocytes and macrophages from the same donor. Monocytes were differentiated to macrophages by culture with M-CSF (20 ng/ml). At day 3, ecteinascidin 743 was added to cultures and incubated for 48 h. Results show the comparison of monocytes and macrophages obtained from the same donor. Viability was assessed by PI staining and analyzed by Flow Cytometry. Similar results were obtained in other 4 experiments. In a series of 4 different experiments, IC50 for in vitro differentiated was 10 nM.

We then tested the susceptibility to ecteinascidin 743 of macrophages classically activated by LPS and IFN gamma (or M1 macrophages) and alternatively activated by IL-4 (or M2 macrophages). In vitro differentiated macrophages were stimulated with LPS (100 ng/ml)+IFNgamma (500 UI:ml), Il-4 (20 ng/ml), in the presence or absence of ecteinascidin 743 for 48 h. Viability was assessed by PI staining and analyzed by Flow Cytometry. Both LPS-stimulated and IL-4-stimulated macrophages were susceptible to drug treatment similarly as non-stimulated macrophages (FIG. 4B).

We also tested Tumour-Associated Macrophages (TAM) isolated from the ascites of non-treated ovarian adenocarcinoma patients. Enriched preparations of TAM isolated from three different patients with ovarian cancer were treated in vitro with ecteinascidin 743 for 48 h. Viability was assessed by PI staining and analyzed by Flow Cytometry. TAM were significantly killed in vitro by ecteinascidin 743 with 40-70% mortality at 10 nM. Results from three different patients are shown in FIG. 4C.

Overall these experiments demonstrate that human mononuclear phagocytes are highly susceptible to the cytotoxic effect of ecteinascidin 743 at concentrations within the therapeutic range. It should be noted that even in the presence of M-CSF, monocytes never underwent cell cycle progression, as checked by DNA analysis with flow cytometry. The toxic effect of ecteinascidin 743 on monocytes is therefore independent from cell cycle and provides the unique opportunity to study the biological effects of this drug on non-replicating cells.

### EXAMPLE 2

### Non-Cytotoxic Concentrations of Ecteinascidin 743 Inhibit In Vitro and In Vivo Macrophage Differentiation

In order to study the effect of ecteinascidin 743 on macrophage differentiation, non cytotoxic doses of the drug were used. Monocytes were cultured with M-CSF (20 ng/ml) and with sub-cytotoxic concentrations of ecteinascidin 743 for 5 days. Phenotype analysis was performed by indirect immunofluorescence and analyzed in Flow Cytometry by gating on large cells. Usually, an average of 65±15% (mean±SD of>10 experiments) of input monocytes differentiate into large cells expressing typical macrophage markers, including CD16, CD68 and CD206 (mannose receptor). After 5 days of culture monocyte viability, evaluated by propidium iodide staining in flow cytometry, was 92% and 70% of untreated cells at 0.5 and 1 nM ecteinascidin 743, respectively. The process of macrophage differentiation was partially inhibited as the de novo expression of CD68, CD16 and CD206 was reduced at 1 nM ecteinascidin 743 (FIG. 3B).

To validate the above in vitro findings we tested whether the in vivo administration of ecteinascidin 743 in tumour patients could have measurable effects on monocyte viability and capacity to macrophage differentiation in vitro. A phase II trial with ecteinascidin 743 is currently underway in advanced ovarian adenocarcinoma patients who had failed two different cycles of conventional cis-platin and taxol-based chemotherapy. Tumour patients selected for this study were treated with 1300 ug/ml/m2 of ecteinascidin 743. Blood samples from patients were drawn just before drug administration and at the end of a 3-hour infusion. Purified monocytes were immediately isolated and cultured with M-CSF (20 ng/ml) for 5 days to induce macrophage differentiation and then analalyzed for phenotype expression. Of 12 evaluable patients, monocytes from 6 subjects showed decreased macrophage differentiation after ecteinascidin 743 treatment. Table 1 shows the phenotype analysis of in vitro differentiated macrophages from patients whose cells after therapy showed at least 50% inhibition of CD206, CD16 and CD68 expression, compared to cells collected before therapy. The data shown are the absolute numbers of marker positive cells for a total of 10,000 input cells. Monocytes collected from the other six patients did not show any significant decrease in their differentiation capacity.

TABLE 1
Effect of in vivo treatment with ecteinascidin 743 on the in vitro
differentiation of macrophages in tumour patients.

| | Absolute numbers of marker | | |
|---|---|---|---|
| | positive macrophages/10,000 cells | | |
| Patients | No exposure | Exposure to ecteinascidin 743 | % inhibition* |
| UPN 1 | | | |
| CD206 | 4350 | 550 | 88 |
| CD16 | 3110 | 1248 | 60 |
| CD68 | 2703 | 1473 | 45 |
| UPN 2 | | | |
| CD206 | 2810 | 595 | 79 |
| CD16 | 2705 | 1105 | 60 |
| CD68 | 3500 | 1060 | 70 |
| UPN 3 | | | |
| CD206 | 3590 | 474 | 87 |
| CD68 | 3260 | 632 | 80 |
| UPN 4 | | | |
| CD16 | 5130 | 3050 | 41 |
| CD68 | 5550 | 2460 | 55 |
| UPN 5 | | | |
| CD16 | 1575 | 594 | 63 |
| CD68 | 1620 | 815 | 50 |
| UPN 6 | | | |
| CD16 | 2750 | 480 | 83 |
| CD68 | 2320 | 505 | 79 |

*% inhibition of macrophage differentiation referred to cells before infusion.

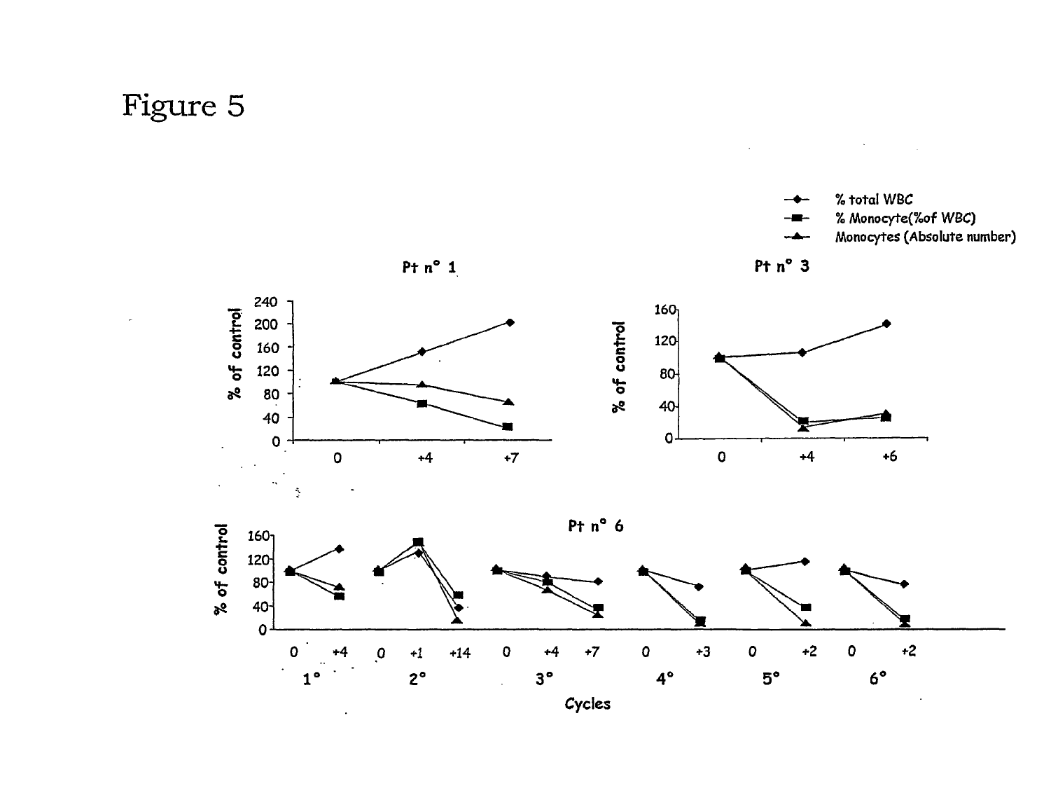

We also investigated whether the in vivo treatment with ecteinascidin 743 caused a measurable monocytopenia in cancer patients. Monocyte values were obtained from blood formula during routine clinical analysis. Of 9 patients whose morphological analysis of monocytes was recorded and able, 7 patients showed a decrease (25% inhibition compared to values available infusion, in at least one cycle) in the number of monocytes, evaluated both as % of monocytes over total leukocytes, and as absolute number of monocytes/ul of blood. Results from three representative patients are shown in FIG. 5. In spite of a constant level or a transient increase in the total number of leukocytes , in the first few days following drug infusion, monocytes never increased and actually were frequently decreased.

### EXAMPLE 3

### Ecteinascidin 743 Inhibits the Production of Inflammatory Cytokines/Chemokines

Monocytes/macrophages are potent producer of soluble factors which orchestrate the inflammatory/immune response. We therefore tested the effect of ecteinascidin 743 treatment on the secretory function of these cells. The chemokine CCL2 is a major chemoattractant for mononuclear phagocytes and is produced by immune as well as several tumour cells. Tumour-derived CCL2 attracts circulating monocytes at the tumour site and the TAM content of a tumour correlates with levels of CCL2, as demonstrated in several tumours.

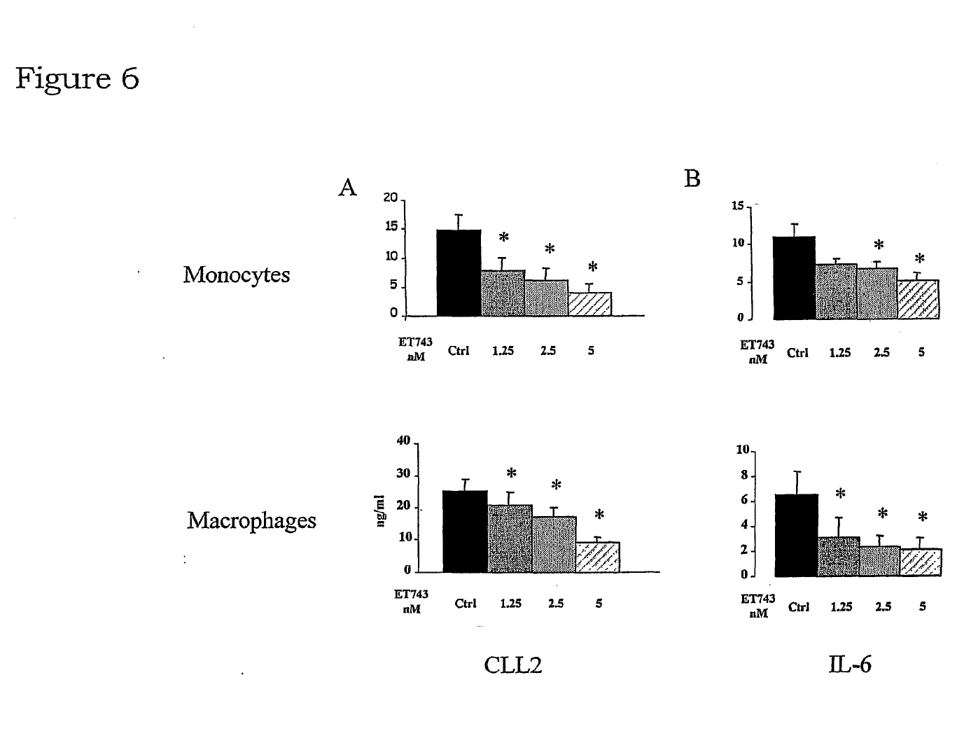

Monocytes and in vitro differentiated macrophages were stimulated with LPS (100 ng/ml). After 1 h LPS stimulation they were treated with ecteinascidin 743. After 16 h incubation, cell supernatants were harvested and tested in ELISA. Under these treatment conditions cell viability was usually >85% for concentrations up to 5 nM. Treatment with ecteinascidin 743 dose-dependently reduced the production of CCL2 by LPS-stimulated monocytes and in vitro-derived macrophages (FIG. 6A). Mean inhibition at 5 nM, for monocytes, was 65% (range 50-80%, n=5) and was 50% (range 25-75%, n=5) for in vitro differentiated macrophages. Results are mean+/−SE of 3-5 experiments

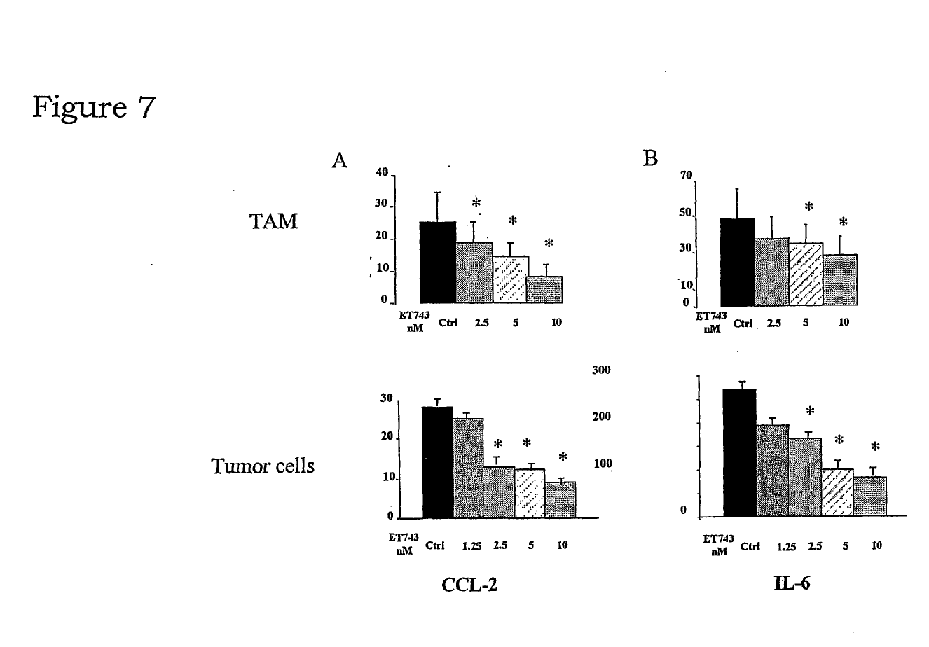

Next, TAM associated to ovarian carcinomas were tested. Freshly isolated ovarian tumor cells and TAM were incubated with ecteinascidin 743 for 16 h. TAM were stimulated with LPS (100 ng/ml). Cell supernatants were harvested and tested in ELISA. Results are mean+/−SE of 4 experiments for TAM and from 1 experiment for tumor cells. The LPS-stimulated production of CCL2 was reduced by 50% (range 40-60%, n=4) (FIG. 7A), while their constitutive production by 43% (range 30-50%, n=4).

We also tested two other cytokines, IL-6 and TNF, produced by macrophages and tumour cells, which have inflammatory properties and also act as growth factors for some tumours. IL-6 production was always reduced after ecteinascidin 743 treatment, with an overall inhibition at 5 nM of 54% (range 51-57%, n=2) and 69% (range 66-72%, n=2), in monocytes and macrophages, respectively (FIG. 6B). IL-6 release in TAM was somehow more resistant to treatment: at 5 nM mean inhibition was 35% (range 25-53%, n=4); at 10 nM was 47% (range 33-63%, n=4), (FIG. 7B).

Of interest, ecteinascidin 743 reduced also the constitutive production of CCL2 and IL-6 by freshly isolated tumour cells. A representative experiment is shown in FIG. 7.

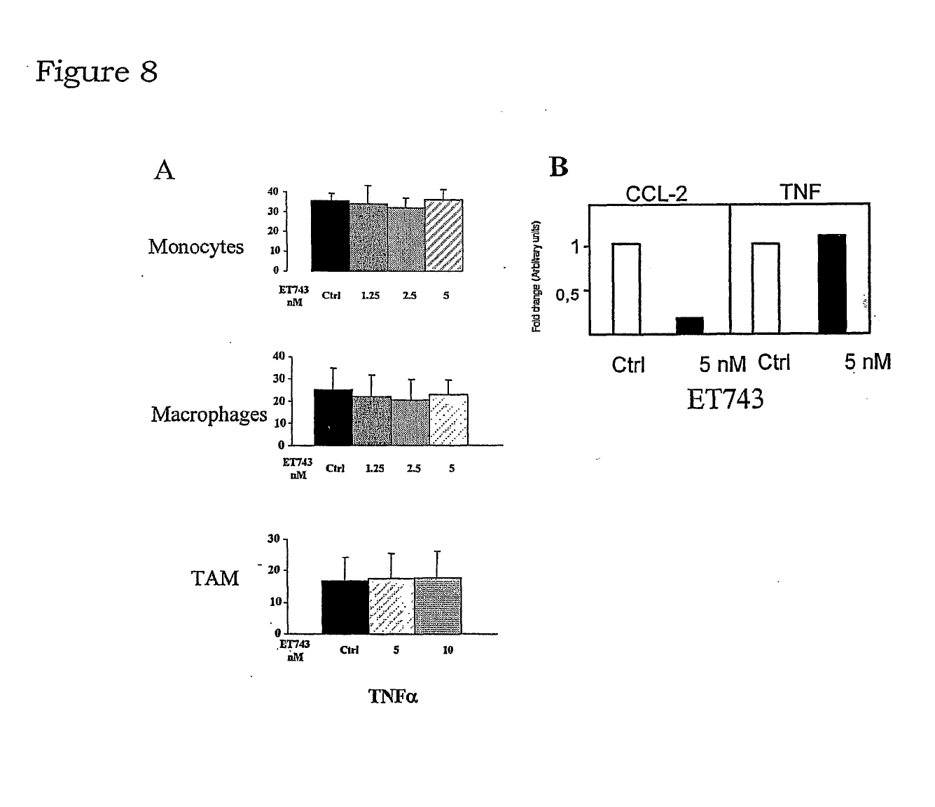

In contrast, and quite surprisingly, when monocytes, in vitro differentiated macrophages and TAM were stimulated with LPS (100 ng/ml), treated with ecteinascidin 743 preceeded of 1 h LPS stimulation, and after 16 h incubation, cell supernatants were harvested and tested in ELISA, it was observed that the production of TNF by monocytes/macrophages, as well as by TAM was never inhibited, even up to 10 nM for TAM (FIG. 8A), suggesting that ecteinascidin 743 interferes only with selected genes. These results also indicate that, under these conditions cells were not damaged by the treatment. To verify whether the inhibitory effect of ecteinascidin 743 on cytokine production was at the transcriptional level, we analyzed mRNA of CCL2 and TNF from LPS-stimulated macrophages by real time-PCR of CCL2 and TNF transcripts in LPS-stimulated monocytes exposed to ecteinascidin 743. As shown in FIG. 8B, after ecteinascidin 743 treatment a consistent reduction of CCL2 transcripts was observed, while TNF mRNA was unaffected, in line with the results obtained in Elisa.

Overall these results indicate that ecteinascidin 743 at pharmacological concentrations reduces the production of two important inflammatory cytokines in mononuclear phagocytes and tumour cells.

### EXAMPLE 4

### Other Ecteinascidin Compounds also Inhibit the Production of Inflammatory Cytokines/Chemokines

We also tested five other ecteinascidin compounds (Table 2) for their capacity to inhibit the production of CCL2 by human monocytes in vitro. Of the five compounds tested, only ET637 Derivative A showed marked and consistent ability to downmodulate inflammatory cytokine production by monocytes, at concentrations of 2.5 and 5 nM. These concentrations did not affect monocyte viability after 48 h of exposure. The extent of inhibition of ET637 Derivative A was even more pronouced compared to ET743. In Table 2 is shown that the production of CCL2, induced by exposure of monocytes to tumor cell supernatants, is inhibited up to 80% and 97% at 2.5 and 5 nM, respectively, in two different donors. In the same experiment ET743 inhibited between 30% and 70%. The other compounds also showed an inhibitory activity, but at a lower level than the other two above mentioned compounds.

TABLE 2
Inhibitory effect of ET743 and other ecteinascidin compounds
on the production of the inflammatory chemotactic cytokine CCL2

| | | Donor A | Donor B |
|---|---|---|---|
| | | % inhibition | % inhibition |
| ET743 | 2.5 nM | 30 | 60 |
| | 5 nM | 70 | 70 |
| ET637 | 2.5 nM | 80 | 80 |
| Derivative A | 5 nM | 97 | 87 |
| ET594 | 5 nM | 25 | — |
| | 10 nM | 25 | — |
| ET743 | 2.5 nM | 30 | — |
| Derivative A | 5 nM | 35 | 30 |
| ET745 | 2.5 nM | — | — |
| | 5 nM | 23 | — |
| ET637 | 2.5 nM | — | — |
| Derivative B | 5 nM | 25 | — |

Similar results were obtained when monocytes were stimulated with LPS (100 ng/ ml) and treated with ET743 and the other ecteinascidin compounds, although the overall inhibition was less marked compared with the previous experiment where the tumor supernantant was used as CCL2-inducing stimulus.

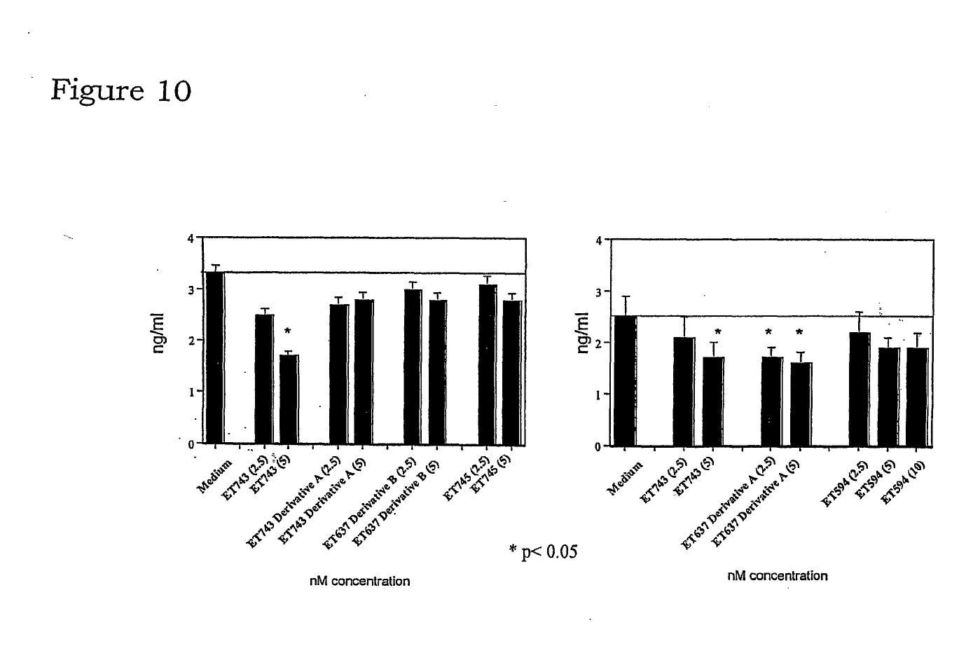

In FIG. 10 it is confirmed that ET637 Derivative A gives a significant inhibition of CCL2 production.

### EXAMPLE 5

Comparison of Ecteinascidin 743 with Antineoplastic Agents Currently Used in Ovarian Cancer

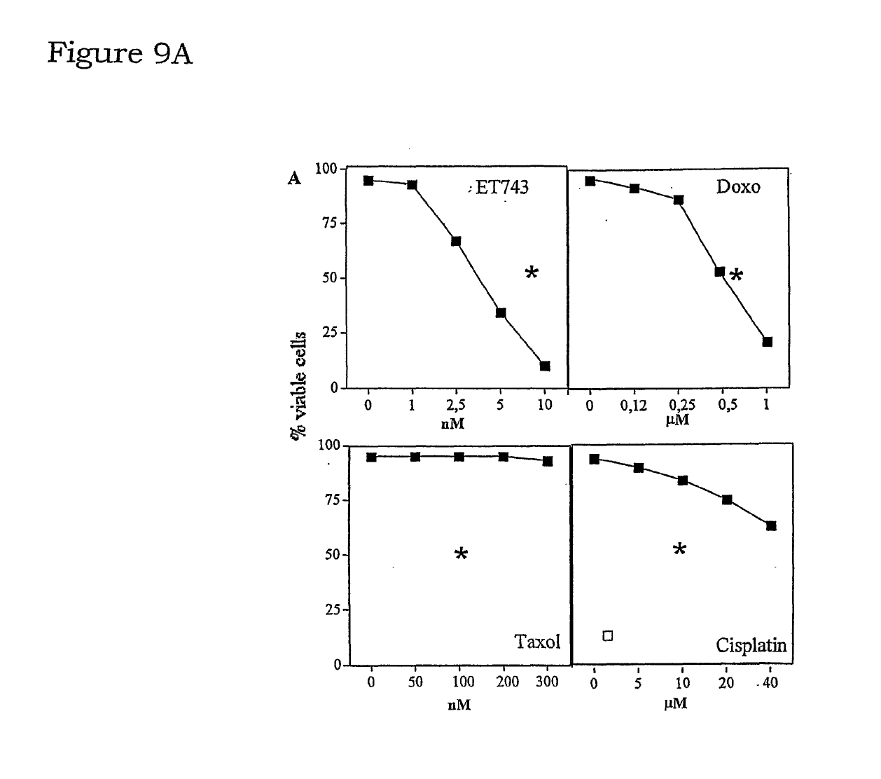

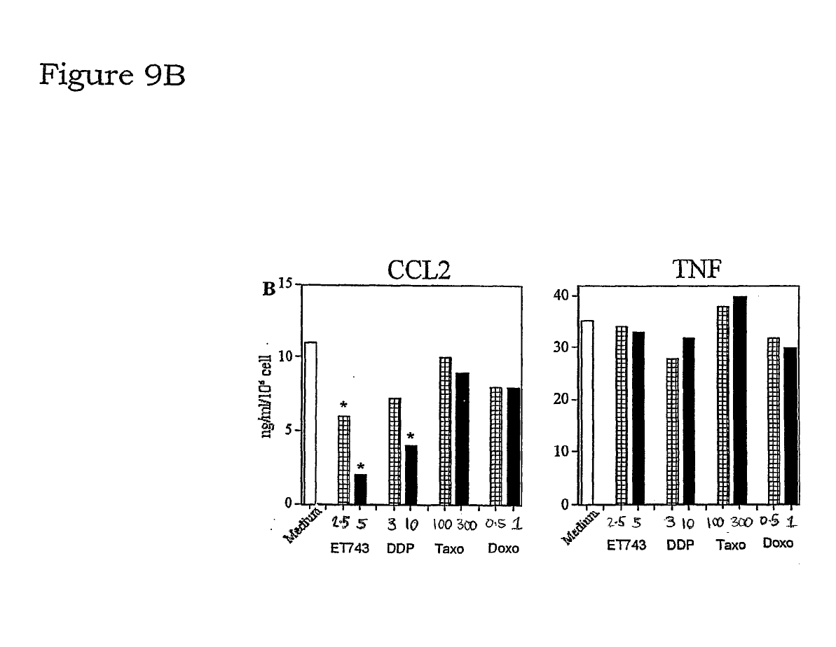

As ecteinascidin 743 is being actively studying for the treatment of ovarian adenocarcinoma, it was of interest to compare these anti-inflammatory effects of ecteinascidin 743 with other compounds conventionally used in this disease, namely Doxorubicin, Cisplatin and Taxol. Monocytes were incubated for 48 h with the indicated concentrations of ecteinascidin 743, Doxorubicin, Taxol and Cisplatin. Viability was assessed by PI staining and analyzed by Flow Cytometry. FIG. 9A shows that at active concentrations on tumour cells (>0.5 μM) Doxorubicin was highly cytotoxic on monocytes after 48 h treatment, while Cisplatin and Taxol were not. Significant toxicity with Cisplatin was observed only at very high concentrations (40 μM), while Taxol was ineffective even at 300 nM.

CCL2 and TNF production by LPS-stimulated monocytes treated with the indicated doses of the anti-tumor agents was also tested. Cell supernatants were harvested after 24 h-incubation and tested in ELISA. As shown in FIG. 9B, Taxol and Doxorubicin were ineffective, but DDP (Cisplatin) (10 μM) reduced CCL2 production. None of these compounds interfered with the production of TNF. These results indicate that monocyte cytotoxicity and inhibition of CCL2 are not generalized properties of anti-tumour agents conventionally used in ovarian cancer treatment.

### DISCUSSION

In this study we have evaluated the cytotoxic effect of ecteinascidin 743 on mononuclear phagocytes. Blood circulating monocytes were highly susceptible to the drug and underwent apoptosis at concentrations of 5 nM/48 h. In vitro differentiated macrophages and Tumour-Associated Macrophages (TAM) were also susceptible at 5-10 nM. These values are within the range of effective therapeutic concentrations. At low concentrations of ecteinascidin 743, monocytes were inhibited in their differentiation to macrophages. We have confirmed these results by studying monocytes from tumour-bearing patients undergoing ecteinascidin 743 therapy. In 6 of 12 patients tested, monocytes collected after 3 h infusion (1300 mg/m2) showed >50% inhibition of in vitro macrophage differentiation compared to monocytes collected just before therapy. Moreover, a significant monocitopenia has been observed in the first few days following drug infusion in the majority of the patients. These results indicate that a brief in vivo exposure to ecteinascidin 743 is sufficient to provide a cytotoxic effect on monocytes.

A major finding of our work is the inhibitory activity of ecteinascidin 743 on the production of inflammatory cytokines. Among various inflammatory cytokines produced by monocyte/macrophages we have tested IL-6, TNF and the chemokine CCL2. CCL2 is a chemokine attracting monocytes and other leukocyte subsets, and is produced both by monocyte/macrophages and several tumour cells. It has been described that ovarian adenocarcinoma cells produce huge amounts of CCL2 and that their levels correlate with the macrophage content of tumours. CCL2 is therefore one of the most important factors regulating monocyte/macrophages recruitment at the tumour site. Ecteinascidin 743 strongly inhibited CCL2 release by LPS-activated monocytes, macrophages and TAM. Ecteinascidin 743 also strongly inhibited the constitutive production of CCL2 by freshly isolated ovarian tumour cells. Thus, lower levels of CCL2 by TAM and tumour cells are likely to reduce the number of macrophages recruited at the tumour site. In the above described in vitro experiments ecteinascidin 743 was present throughout the 16-h culture period. We also checked whether a shorter in vitro exposure to ecteinascidin 743 was sufficient to affect cytokine production. Monocytes exposed to ecteinascidin 743 were washed after 1 hour culture and replaced in fresh medium. Under these conditions, inhibition of CCL2 production was still significant, though slightly lower compared to cells receiving 16 h-treatment (57% and 69% inhibition, respectively).

IL-6 is a pro-inflammatory cytokine with important effects on the immune/hematopoietic system and is a co-factor for the production of CCL2. In addition, several studies have pointed out that IL-6 may act as a growth factor for some tumour cells, including ovarian cancer. As for CCL2, the LPS-induced IL-6 was dramatically decreased in monocytes /macrophages by ecteinascidin 743. The constitutive IL-6 production of freshly isolated ascitic tumour cells was also reduced.

A novel, recently described effect of IL-6 is its ability to rescue T lymphocytes from the regulatory T cells (Treg)-mediated suppression. Treg are a small, albeit very important subset of T lymphocytes which control T cell auto-reactivity and maintain homeostasis. A role for Treg in auto-immune disease is well recognized. Auto-reactive T lymphocytes suppressed by Treg can be rescued by IL-6, thus perpetuating the auto-immune reaction. Therefore, the ecteinascidin 743-mediated reduction of IL-6 could be a favourable therapeutic effect. Ecteinascidin 743 has never been considered for the treament of chronic inflammatory disorders. The results of this study point out that both for its cytotoxic effect on precursors of antigen presenting cells (i.e. monocytes) and for its ability to decrease IL-6, ecteinascidin 743 is an interesting candidate in anti-inflammatory therapy.

Unlike CCL2 and IL-6, ecteinascidin 743 had no significant effect on the production of TNF, another important inflammatory mediator, produced by LPS-stimulated monocyte/macrophages.

We have demonstrated that other ecteinascidin compounds as well as ET743 are able to inhibit the production of CCL2 by human monocytes. From the compounds tested, ET637 Derivative A has showed marked and consistent ability to downmodulate CCL2 production. The extent of inhibition of ET637 Derivative A was even more pronouced compared to ET743. The other compounds also showed an inhibitory activity, but at lower levels.

In conclusion, the finding that ecteinascidin 743 and the other ecteinascidin compounds affect viability and functions of monocyte/macrophages discloses novel effects of these compounds and a new therapeutic indication.

1. A method of treating inflammation which comprises administration of an effective amount of an ecteinascidin compound of general formula (I): 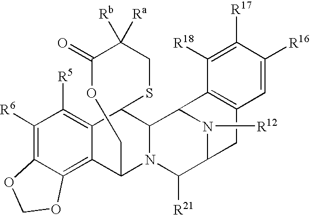
wherein:
R⁵ is OH, alkoxy or alkanoyloxy;
R⁶ is hydrogen, alkyl, alkenyl, alkynyl or aryl;
R¹² is hydrogen, alkyl, alkenyl, alkynyl or aryl;
R¹⁶ is hydrogen, alkyl, alkenyl, alkynyl or aryl;
R¹⁷ is OH, alkoxy or alkanoyloxy;
R¹⁸ is OH, alkoxy or alkanoyloxy;
R²¹ is H, OH, CN or another nucleophilic group; and
R^{a} is hydrogen and R^{b} is optionally substituted amino, or
R^{a} with R^{b} form a carbonyl function ═O, or
R^{a}, R^{b} and the carbon to which they are attached form a tetrahydroisoquinoline group.

2. The method according to claim 1, wherein the inflammation is caused by a disease selected from the group consisting of chronic inflammatory diseases, autoimmune diseases and atherosclerosis.

3. The method according to claim 1, wherein in the ecteinascidin compound of formula (I), the group R⁵ is an alkanoyloxy.

4. The method according to claim 1, wherein in the ecteinascidin compound of formula (I), the group R⁶ is methyl.

5. The method according to claim 1, wherein in the ecteinascidin compound of formula (I), the group R¹² is methyl.

6. The method according to claim 1, wherein in the ecteinascidin compound of formula (I), the group R¹⁶ is methyl.

7. The method according to claim 1, wherein in the ecteinascidin compound of formula (I), the group R¹⁷ is methoxy.

8. The method according to claim 1, wherein in the ecteinascidin compound of formula (I), the group R¹⁸ is OH.

9. The method according to claim 1, wherein in the ecteinascidin compound of formula (I), the group R²¹ is H, OH or CN; and
R^{a} is hydrogen and R^{b} is an amido group, or
R^{a} with R^{b} form ═O, or
R^{a}, R^{b} and the carbon to which they are attached form a group of formula (II):

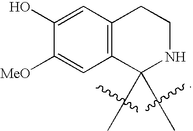

10. The method of claim 1, wherein the ecteinascidin comopund is of formula (III): 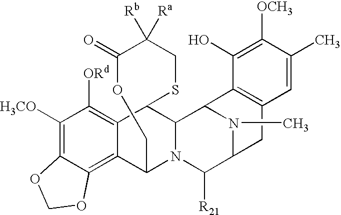
where
R^{a} is hydrogen and R^{b} is amido of formula 13 NHR^{f}— where R^{f} is alkanoyl, or
R^{a} with R^{b} form ═O, or
R^{a}, R^{b} and the carbon to which they are attached form a group of formula (II): 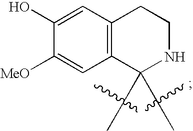
R^{d} is alkanoyl; and
R²¹ is H, OH or CN.

11. The method of claim 10, wherein the ecteinascidin compound is selected from the group consisting of:

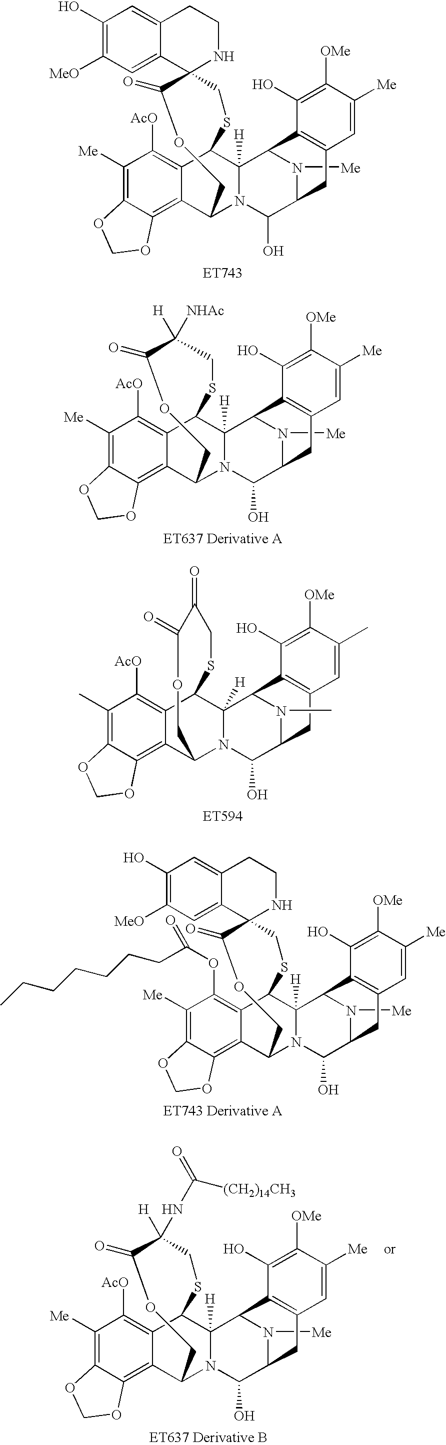

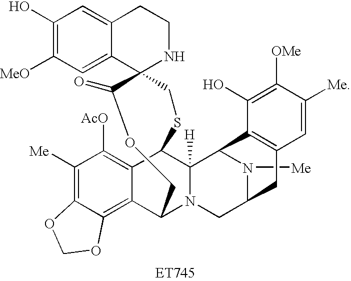

12. The use of an ecteinascidin compound of general formula (I): 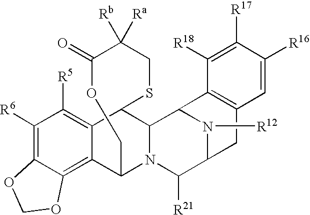
wherein:
R⁵ is OH, alkoxy or alkanoyloxy;
R⁶ is hydrogen, alkyl, alkenyl, alkynyl or aryl;
R¹² is hydrogen, alkyl, alkenyl, alkynyl or aryl;
R¹⁶ is hydrogen, alkyl, alkenyl, alkynyl or aryl;
R¹⁷ is OH, alkoxy or alkanoyloxy;
R¹⁸ is OH, alkoxy or alkanoyloxy;
R²¹ is H, OH, CN or another nucleophilic group; and
R^{a} is hydrogen and R^{b} is optionally substituted amino, or
R^{a} with R^{b} form a carbonyl function ═O, or
R^{a}, R^{b} and the carbon to which they are attached form a tetrahydroisoquinoline group in the preparation of a medicament for use in a method according to any preceding claim.

13. A medicament for treatment of inflammation comprising an ecteinascidin compound of general formula (I): 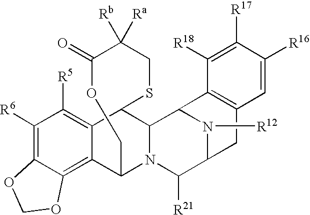
wherein:
R⁵ is OH, alkoxy or alkanoyloxy;
R⁶ is hydrogen, alky, alkenyl, alkynyl or aryl;
R¹² is hydrogen, alky, alkenyl, alkynyl or aryl;
R¹⁶ is hydrogen, alkyl, alkenyl, alkynyl or aryl;
R¹⁷ is OH, alkoxy or alkanoyloxy;
R¹⁸ is OH, alkoxy or alkanoyloxy;
R²¹ is H, OH, CN or another nucleophilic group; and
R^{a} is hydrogen and R^{b} is optionally substituted amino, or
R^{a} with R^{b} form a carbonyl function ═O, or
R^{a}, R^{b} and the carbon to which they are attached form a tetrahydroisoquinoline group, and a pharmaceutically acceptable carrier.
